(19) United States
(12) Patent Application Publication
(10) Pub. No.: US 2005/0031662 A1
(43) Pub. Date: Feb. 10, 2005

(54) Bioreactive methods and devices for treating uterine bleeding

(75) Inventors: Paul Danielson, Shakopee, MN (US); Douglas J. Duchon, Chanhassen, MN (US)

Correspondence Address: INSKEEP INTELLECTUAL PROPERTY GROUP, INC, 1225 W. 190TH STREET, SUITE 205, GARDENA, CA, 90248, US

(73) Assignee: ImPres Medical, Inc.

(21) Appl. No.: 10/851,364

(22) Filed: May 21, 2004

Related U.S. Application Data
(63) Continuation-in-part of application No. 10/726,433, filed on Dec. 3, 2003.
Application No. 10/726,433 is a continuation of application No. 09/840,951, filed on Apr. 24, 2001, now Pat. No. 6,708,056.
(60) Provisional application No. 60/472,643, filed on May 21, 2003.

(51) Int. Cl. A61B 6/00

(52) U.S. Cl. 424/422; 600/431

(57) ABSTRACT

The present invention contemplates a biocompatible and bioreactive process and device that causes a tissue response in the uterus of a female so as to treat a bleeding pattern. Such a tissue response is bioreactive in nature and substantially noninflammatory. It causes migration of myometrial tissue into the implant and deactivates the normal interrelated cycle of endometrial tissue recycling.

### RELATED APPLICATIONS

This application is a non-provisional application that claims priority to U.S. Provisional Patent Application Ser. No. 60/472,643 entitled Bioreactive Methods And Devices For Treating Abnormal Bleeding filed May 21, 2003. This application is also a continuation-in-part of U.S. application Ser. No. 10/726,433 filed Dec. 3, 2003 entitled Method And Apparatus For Creating Intrautenne Adhesions which is a continuation of U.S. application Ser. No. 09/840,951 filed Apr. 24, 2001 entitled Method And Apparatus For Creating Intrauterine Adhesions. All of the above applications are incorporated herein by reference.

### FIELD OF THE INVENTION

The present invention relates to treating uterine bleeding in females. The present invention particularly relates to treating a uterine bleeding condition in female patients.

### BACKGROUND OF THE INVENTION

As stated in the above-referenced related patent applications, disruptions to the uterine wall and uterine tissue and, in some instances, the formation of intra uterine adhesions (IUA) are believed to be beneficial in creating amenorrhea in women with normal or abnormal menstrual, dysfunctional, neoplastic, infectious, or inflammatory uterine bleeding (e.g., menorrhagia). However, what has remained elusive is a verified determination of the particular type of treatment that will lead to the best desired result.

It has been conventional thinking in the prior art that implants of a biocompatible nature (e.g., implants comprised of Prolene) are the implants best suited for achieving this desired result. However, such prior art implants have introduced ambiguity insofar as the results achieved with such prior art implants are not always consistent. For example, a result observed with one biocompatible implant material in the uterus of one subject may be different than the result observed using the same or substantially the same biocompatible implant in the uterus of a different subject. Both results may be sufficient to achieve the desired result but the results themselves are not entirely identical. These prior art implants may thus be suitable for treating the target condition, however, the differing results makes it difficult to arrive at an implant that is optimal for all candidates under all, or substantially all, conditions.

In view of the above, there is a need to arrive at an implant and an implant protocol that decreases or even eliminates the unpredictability with prior art implants. In particular, there is a need to arrive at a method and device (e.g., an implant) that may be presented to patients as being consistently successful and repeatable across a wide segment of potential patients. In addition, the treatment method and device should reduce patient recovery times and hospital costs. Overall, the method of treatment should also improve the quality of life for patients, particularly insofar as predictability is enhanced.

### OBJECTS AND SUMMARY OF THE INVENTION

In view of the foregoing, it is an object of the present invention to overcome the aforestated limitations of the prior art.

It is a further object of the present invention to present a method and device that generates a response in the uterus that adequately and reliably treats a bleeding condition of a female.

It is a further object of the present invention to present a method and device that functions to produce a consistent result over a wide range of patients and conditions.

It is a further object of the present invention to provide an implant that is safe and reliable and cost effective for users.

These and other objects not specifically enumerated here are believed to be addressed by the present invention which contemplates the use of not only a biocompatible implant but an implant that is also bioreactive in nature. More specifically, the present invention contemplates a bioreactive implant that generates a consistent and reproducible response in the uterus of a female patient so as to provide a reliable treatment mechanism to establish amenorrhea.

### BRIEF DESCRIPTION OF THE DRAWINGS

Features and advantages of the present invention that are believed to achieve the aforesaid objects will be evident from the following description of particular embodiments in conjunction with the drawings, in which:

FIG. 1 is a histological photomicrograph of a tissue response to the presence of a bioreactive implant in the uterus of an animal;

FIG. 2 is an enlarged histological photomicrograph of a specified region of FIG. 1;

FIG. 3 is an enlarged histological photomicrograph of a specified region of FIG. 1;

FIG. 4 is an enlarged histological photomicrograph of a specified region of FIG. 1;

FIG. 5 is an enlarged histological photomicrograph of a specified region of FIG. 1;

FIG. 6 is an enlarged histological photomicrograph of a specified region of FIG. 1;

FIG. 7 is an enlarged histological photomicrograph of a specified region of FIG. 1;

FIG. 8 is an enlarged histological photomicrograph of a specified region of FIG. 1;

FIG. 9 is an enlarged histological photomicrograph of a specified region of FIG. 1;

FIG. 10 is a histological photomicrograph of a tissue response to the presence of a bioreactive implant in the uterus of a female human;

FIG. 11 is a histological photomicrograph of a tissue response to the presence of a bioreactive implant in the uterus of a female human;

FIG. 12 is a histological photomicrograph of a tissue response to the presence of a bioreactive implant in the uterus of a female human;

FIG. 13 is a histological photomicrograph of a tissue response to the presence of a bioreactive implant in the uterus of a female human;

FIG. 14 is a histological photomicrograph of a tissue response to the presence of a bioreactive implant in the uterus of a female human;

FIG. 15 is a histological photomicrograph of a tissue response to the presence of a bioreactive implant in the uterus of a female human;

FIG. 16 is a histological photomicrograph of a tissue response to the presence of a bioreactive implant in the uterus of a female human; and,

FIG. 17 is a histological photomicrograph of a tissue response to the presence of a bioreactive implant in the uterus of a female human.

### DETAILED DESCRIPTION OF THE INVENTION

Recent discoveries by the inventors indicate that the use of a process and/or material that is not merely biocompatible but is also bioreactive in some fashion with the tissue in the uterus in fact produces the desired result to treat the bleeding condition. In the past, prior art implants have been more generally targeted as being only biocompatible in nature. As a result the bioreactive response to the implant has not been discovered as being a key component to reliable and repeatable treatment.

In this regard, the inventors have determined that this bioreactive response may take the form of a proliferative response, a fibroproliferative reaction or an inducement of fibrosis. The response may also manifest itself in the form of angiogenesis, fibroblast migration or metaplasia. Furthermore, it has been determined that this bioreactive response may occur in the intrauterine cavity or the myometrium of the uterus. However, the common denominator is that the implant or process results in a bioreactive tissue response that treats the bleeding condition of the patient.

In this regard, in a preferred embodiment, the inventors have discovered that an implant comprised of polyester (e.g., polyethylene terephthalate or “PET”) induces the desired bioreactive response in the uterus (although the invention is not limited to the use of PET). More specifically, the inventors have discovered that polyester (e.g., PET) in the form of a velour knit leads to the desired response.

For example, a polyester knit implanted into the uterus of a mammal for a period of time (e.g., 30 days), will lead to an alteration of the cells of the uterus in a fashion such that a targeted bleeding condition in the uterus is prevented. There is no apparent need for the polyester knit implant to be loaded with a drug or other cell population to induce alteration of cells necessary to achieve this result.

More specifically, the polyester knit produces a fibroproliferative reaction that 1) eliminates the presence of endometrial cells; 2) stimulates the invagination of the polyester knit by cells originating in the myometrium (e.g., fibroblasts, histiocytes, myocytes, stromal cells, giant cells and related cells) moving from a normal body space, the myometrium, into the knit which is placed in the interior of the uterine cavity; and 3) affixes the polyester knit to the wall of the uterine cavity thereby obliterating the normal physiological relationship and function of the endometrium-myometrium which typically leads to monthly uterine bleeding.

A material that is only biocompatible in nature does not eliminate normal cells, does not stimulate movement of other normal cells into entirely different body compartments and does not obliterate physiological function. Hence, it is evident that a material that is biocompatible and nothing more does not provide the necessary characteristics to achieve the desired goal.

Using a bioreactive material as described above also simplifies the treatment process since the material needs no other cells or substances to stimulate the desired response. In other words, using a bioreactive substance enables the treatment to occur solely through the use of endogenous uterine cells. No exogenous cells or substances are necessary to induce the desired response from the native uterine cells. However, it is within the scope of the invention to utilize a substance on the bioreactive material that may control and direct the bioreactive response inherent in the material if so desired.

Substances other than polyester (e.g., PET) are within the scope of the invention. Any medical grade material that is used to promote tissue ingrowth is a candidate for treating bleeding in the uterus in accordance with the present invention. This could include PTFE or other fibrous polyurethane substances, including meshes. One particular example of an appropriate candidate is the type of materials used for cardiovascular fabrics, surgical felts, pledgets and tapes offered by Bard and disclosed in related promotional material published by Bard, all of which is incorporated herein by reference.

However, it is emphasized that the material and process in accordance with the present invention must be of the nature such that native uterine cells react to the material. Materials and/or processes that readily receive or that are readily acceptable to the uterine cells, i.e., materials and processes that do not cause a cellular reaction as discussed above, are unlikely to lead to the desired anti-bleeding response within the uterus.

In further description of preferred embodiments of the present invention, the inventors provide the following examples demonstrating the positive attributes of the present invention.

Studies: Animal and Human

The inventors implanted a PET substance (i.e., Dacron) into the uterus of several sheep for a period of around 21 days. The inventors also implanted a PET substance (i.e., Dacron) into the uterus of several human female patients for a period of around 90 days. In each of these studies, the inventors first disrupted the endometrial tissue so that the Dacron substance was at least partially in contact with myometrial tissue at the time of implantation.

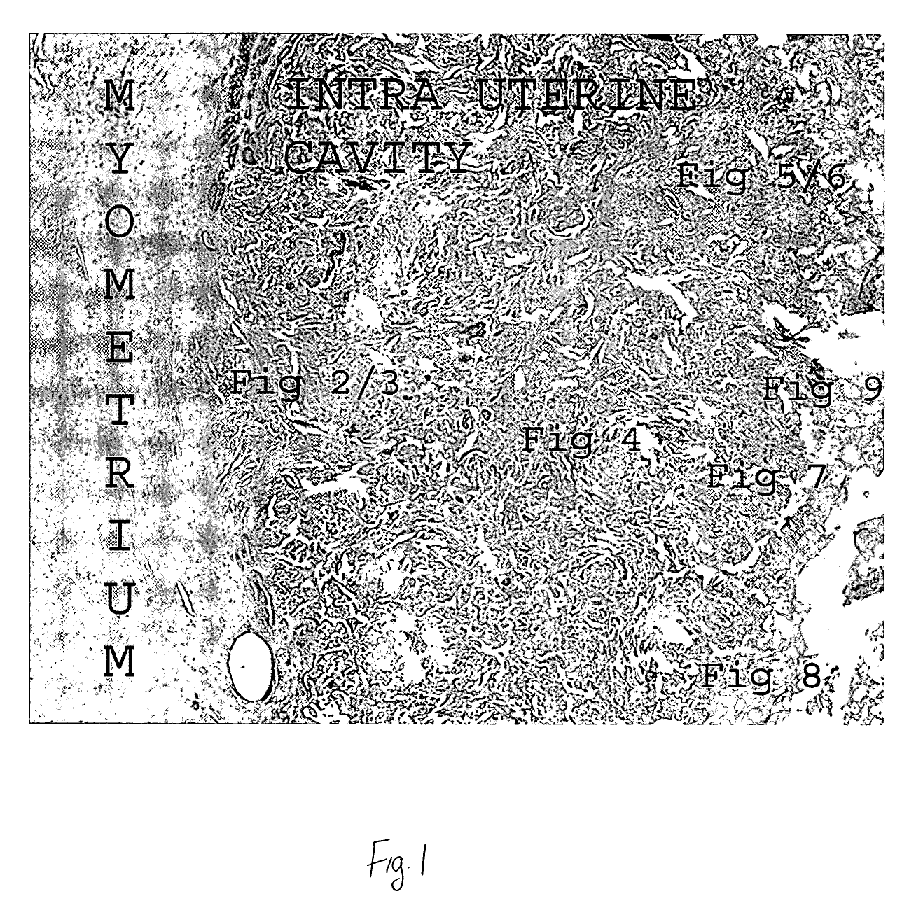

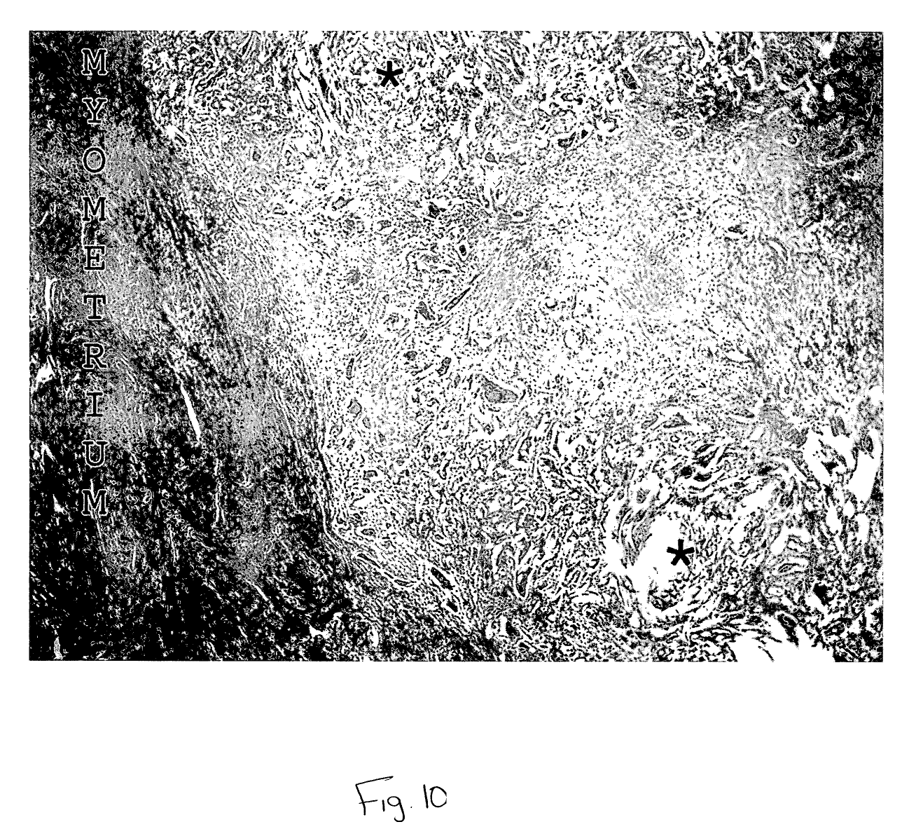

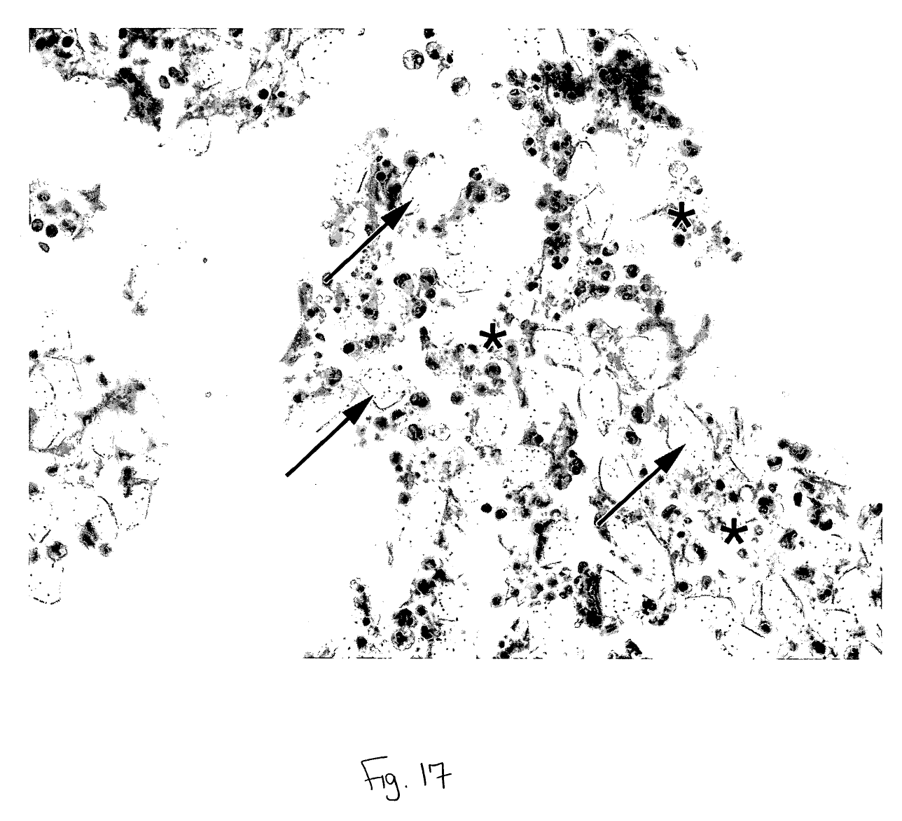

The photomicrographs provided herewith show the histology of the uterine tissue from both the sheep subjects (FIGS. 1-9) and the human female subjects (FIGS. 10-17) of the above-referenced studies. Both sets of photomicrographs confirm the bioreactive nature of the PET substance (in this case a Dacron mesh) and confirm the desired result in accordance with the present invention.

In this regard, although the period of implantation in each of the above-referenced studies is different, the inventors have performed a similar study in sheep and humans where the implant period was around 30 days for both. The results of those studies were consistent with the studies reflected in the Figures discussed below.

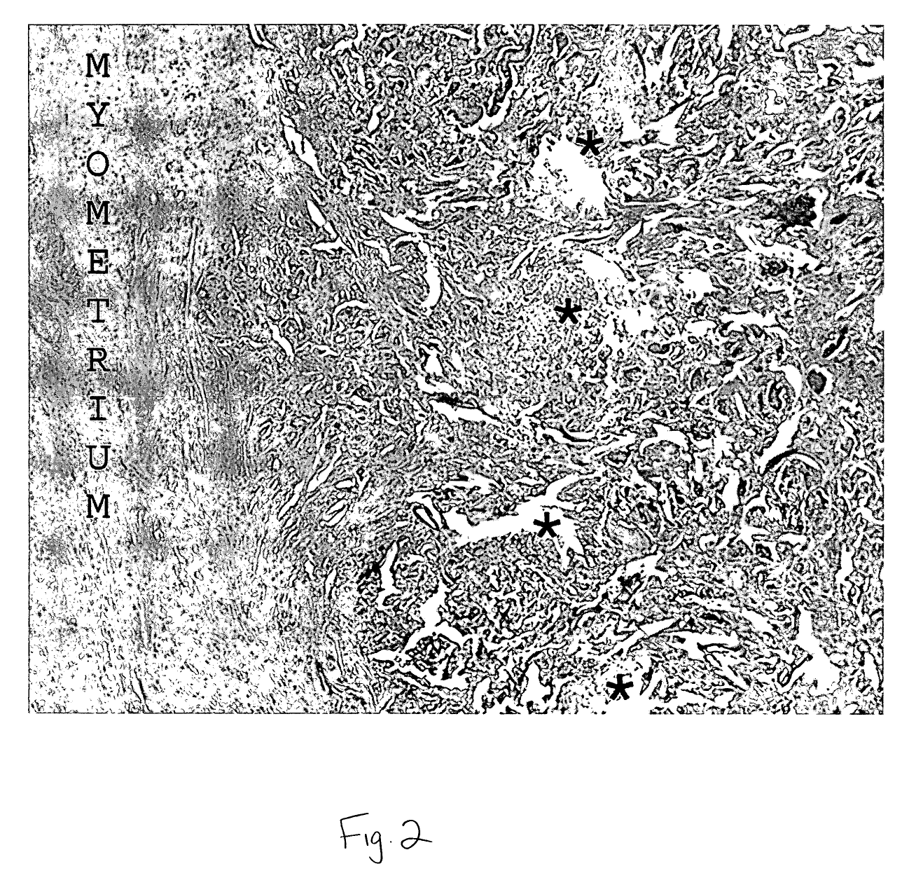

Referring to FIG. 1, a 20× magnification cross section of uterine tissue from the sheep study is shown. It depicts the progression of intrauterine fibroproliferative response to the Dacron in accordance with the present invention. More specifically, the left side of FIG. 1 depicts myometrial tissue followed to the right by mature and remodeled base uterine tissue that has resulted from the reaction to the Dacron. Traversing further to the right of the figure, one can see earlier stages of the reaction until reaching the furthermost right side of the Figure which shows the leading edge (and therefore the earliest stage) of the reaction. FIG. 1 also depicts the regions of FIG. 1 which were magnified and photographed to serve as FIGS. 2-9 which are discussed below.

FIG. 2 is a 40× magnification of the FIG. 2 region of FIG. 1 and shows unidirectional intrauterine fibroproliferative response in accordance with the present invention in a region with myometrial wall contact. Polyester fibers composed of numerous microfilaments make up the Dacron fabric center and are seen passing through the fibroproliferative response. Representative polyester fibers are designated with an asterisk, *, on FIG. 2. These microfilaments are also present separately within the fibroproliferative response that makes up the fabric surface. This response was also observed in the human study as depicted in FIG. 10 which is a 40× magnification of a region of human uterine tissue analogous to the region depicted in FIG. 2 of sheep uterine tissue.

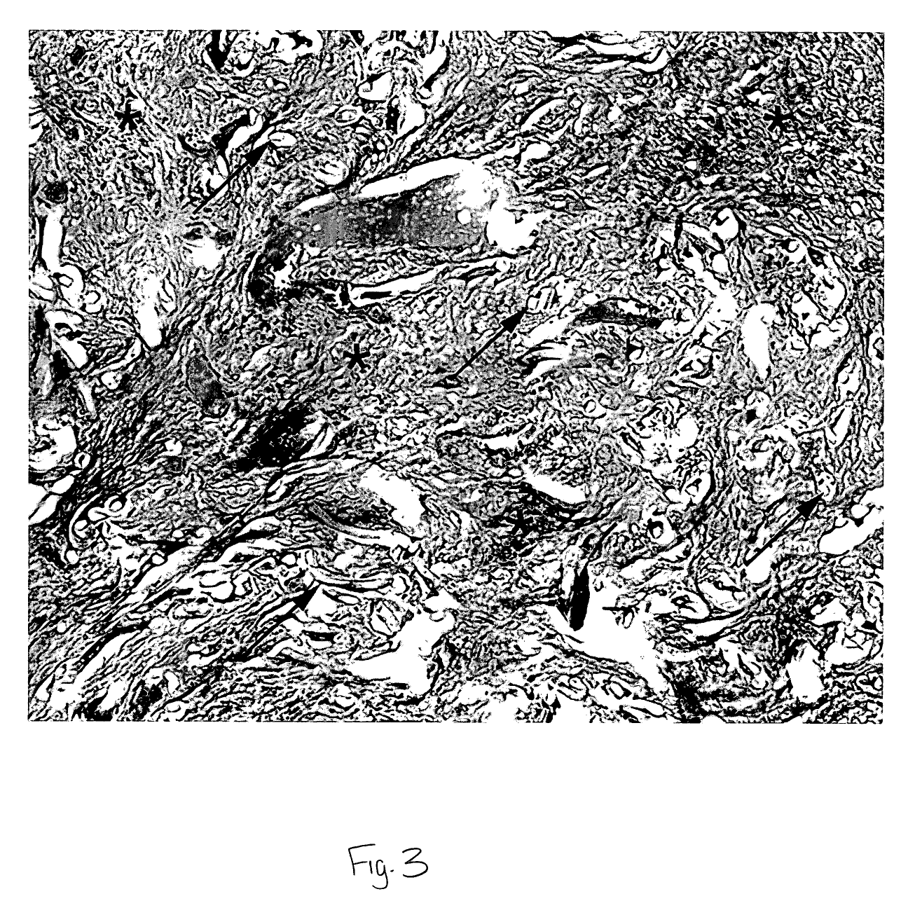

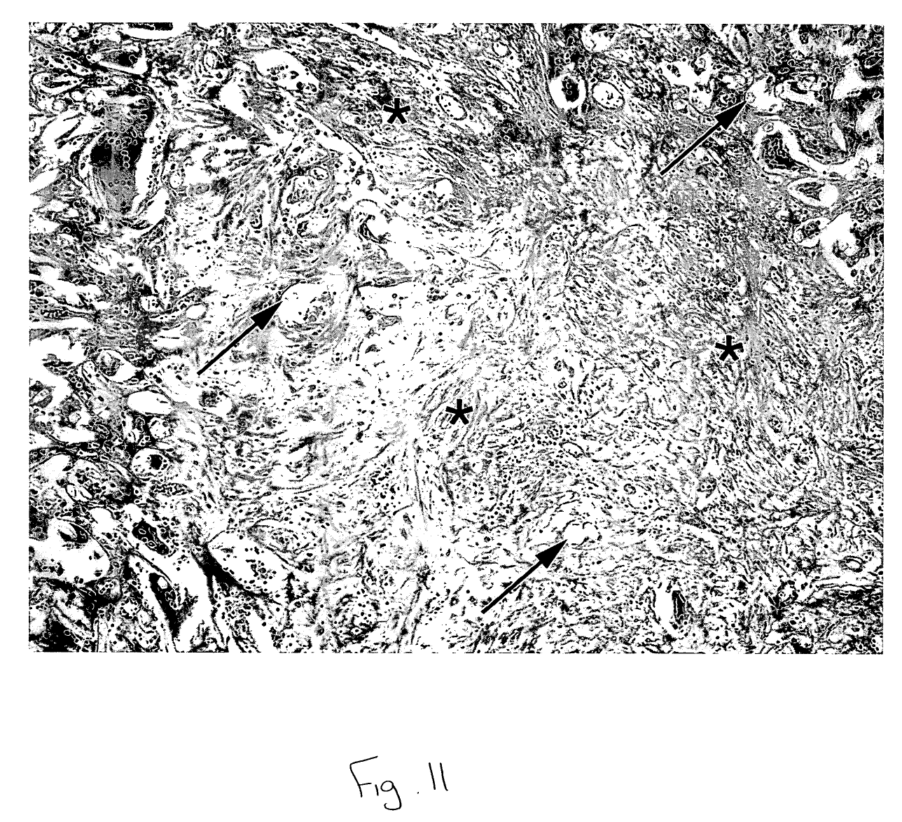

FIG. 3 is a 100× magnification of the FIG. 3 region of FIG. 1 and shows collagen deposition, *, within the fibroproliferative response. Microfilaments of polyester, as depicted by the arrows, are seen interspersed within the tissue. This response was also observed in the human study as depicted in FIG. 11 which is a 100× magnification of a region of human uterine tissue analogous to the region depicted in FIG. 3 of sheep uterine tissue.

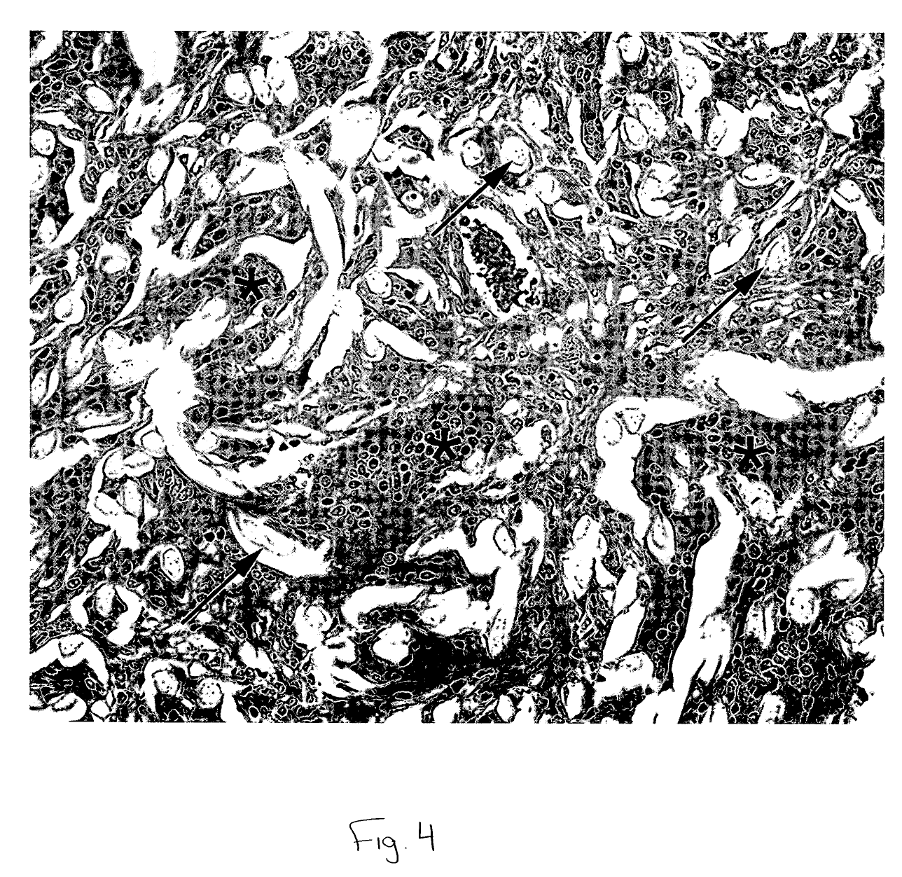

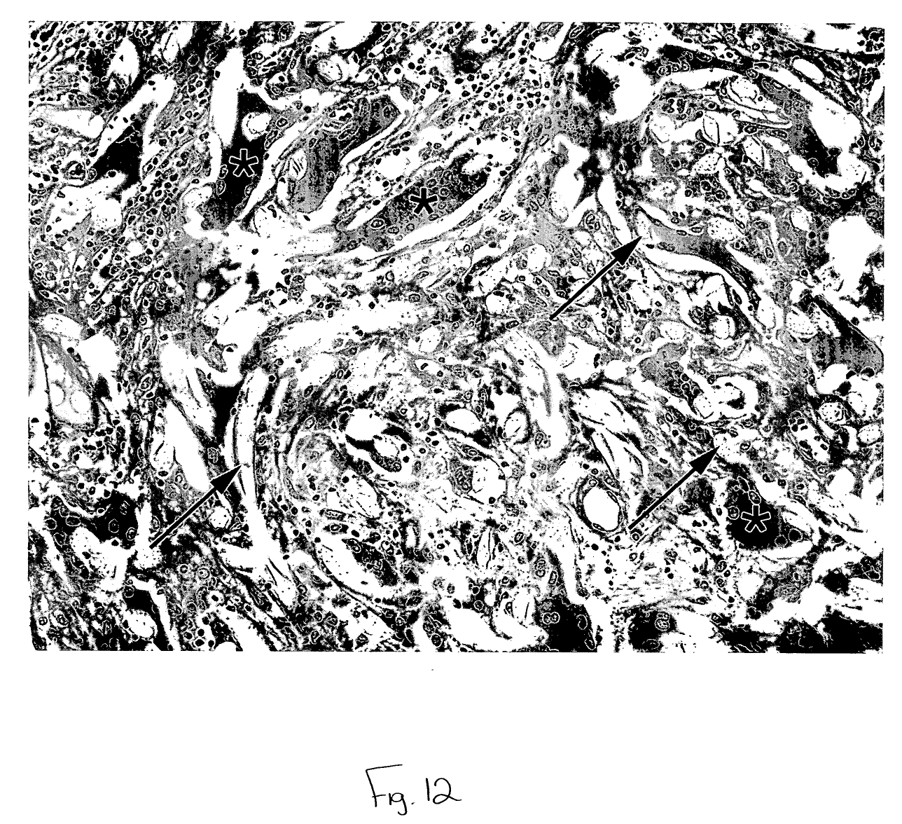

FIG. 4 is a 200× magnification of the FIG. 4 region of FIG. 1 and shows multinucleated giant cells, *, and histiocytic response intermixed with the fibroproliferative response. Multiple microfilaments of Dacron are present as indicated by the arrows. This response was also observed in the human study as depicted in FIG. 12 which is a 200× magnification of a region of human uterine tissue analogous to the region depicted in FIG. 4 of sheep uterine tissue.

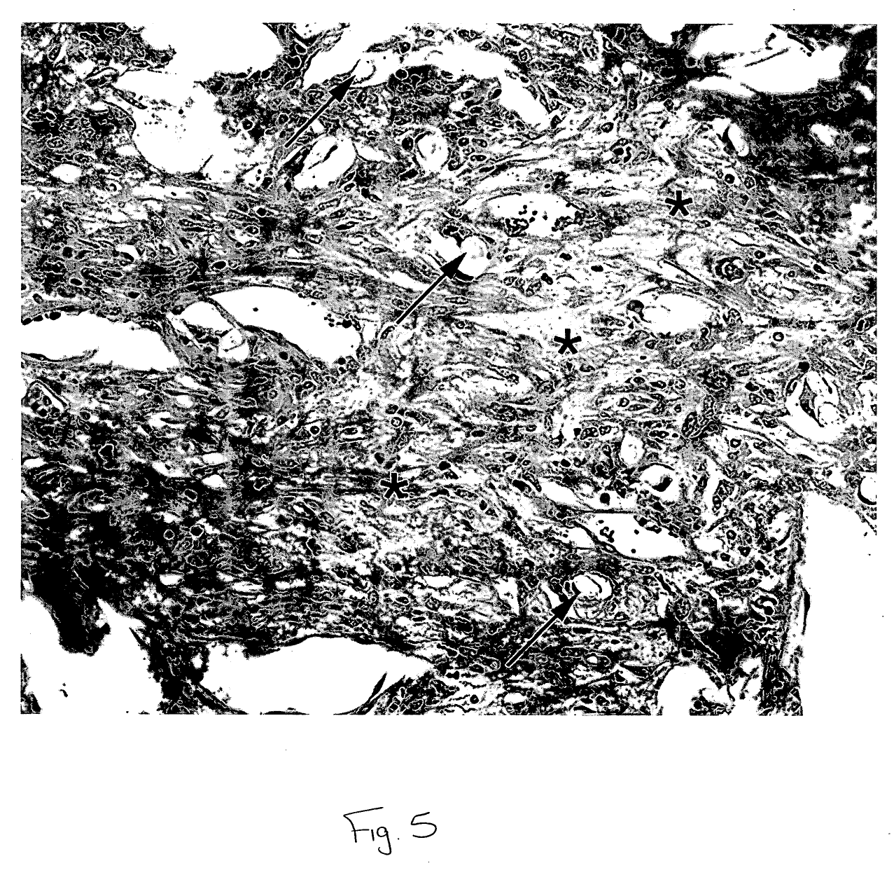

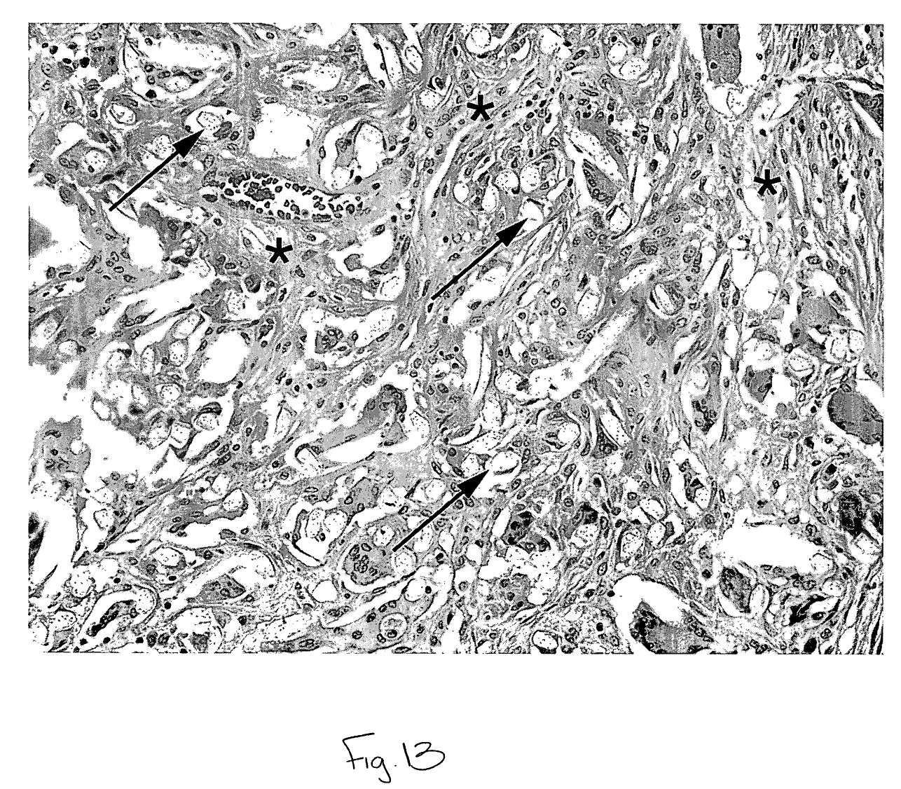

FIG. 5 is a 200× magnification of the FIG. 5 region of FIG. 1 and shows with an asterisk, *, the fibroproliferative response in a region closer to the leading edge of the reaction of new tissue ingrowth. As indicated by arrows, multiple microfilaments of Dacron are seen coursing through the fibroproliferative response. This response was also observed in the human study as depicted in FIG. 13 which is a 200× magnification of a region of human uterine tissue analogous to the region depicted in FIG. 5 of sheep uterine tissue.

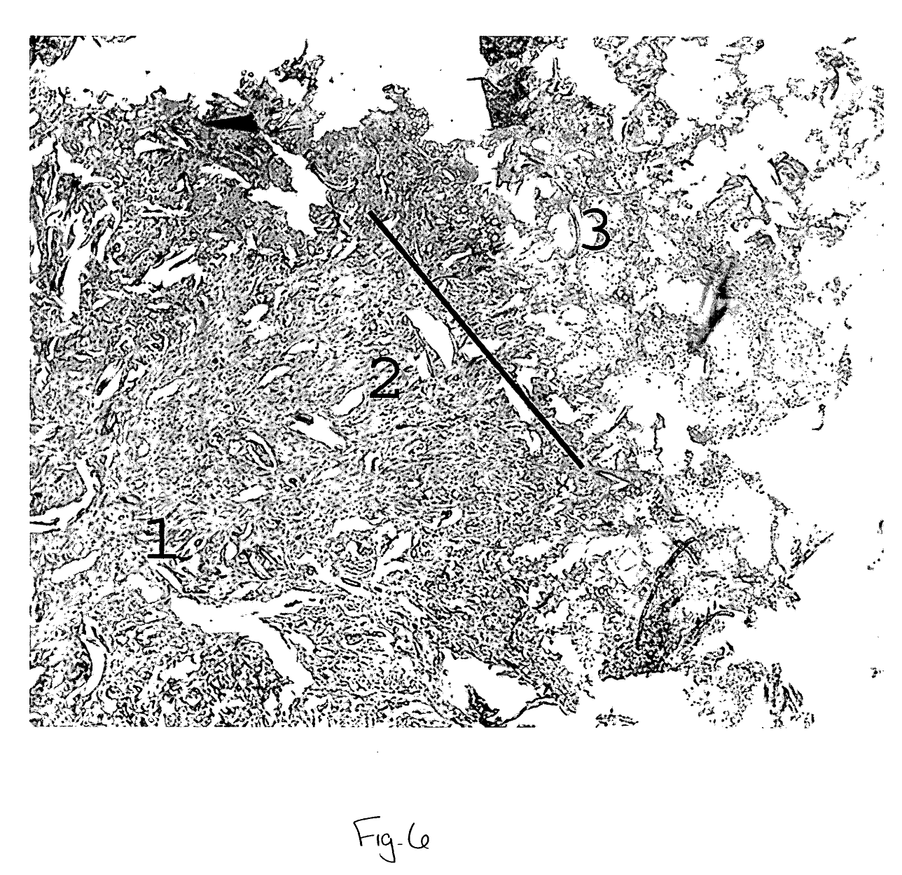

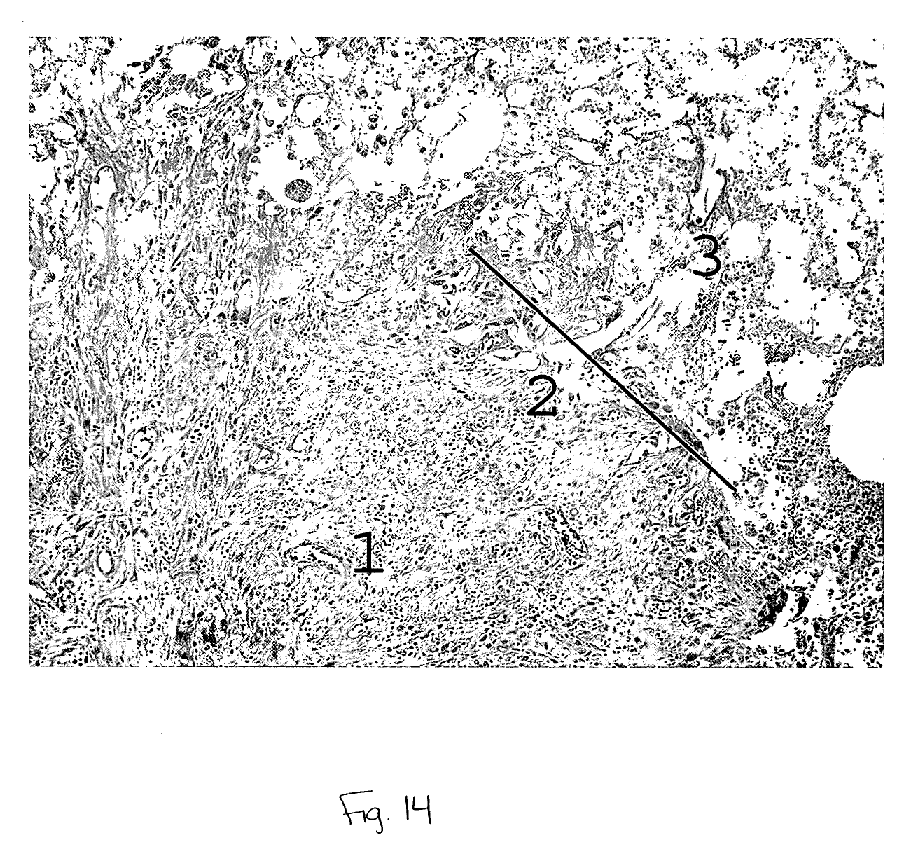

FIG. 6 is a 100× magnification of the FIG. 6 region of FIG. 1 and shows a black bar that generally indicates the edge of new tissue ingrowth of the fibroproliferative reaction. As delineated by the numerals present on the figure, this view also shows: collagen deposition 1, granulation tissue 2, and serous fluid, fibrin and degenerating cells, 3. This response was also observed in the human study as depicted in FIG. 14 which is a 100× magnification of a region of human uterine tissue analogous to the region depicted in FIG. 6 of sheep uterine tissue.

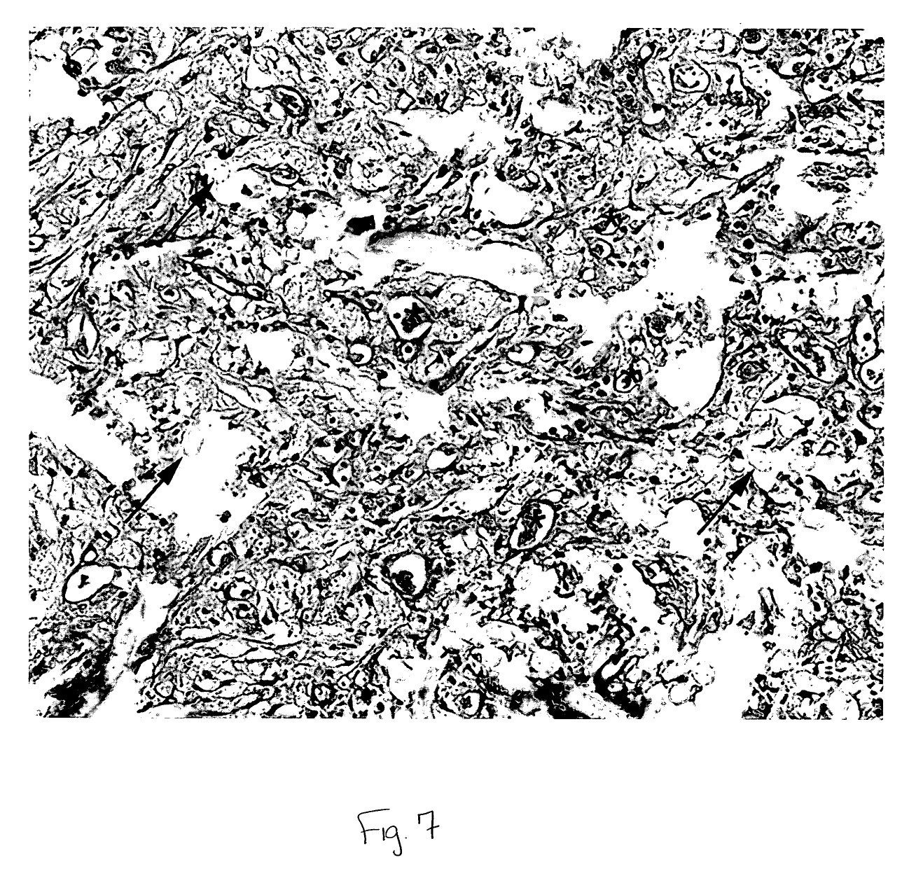

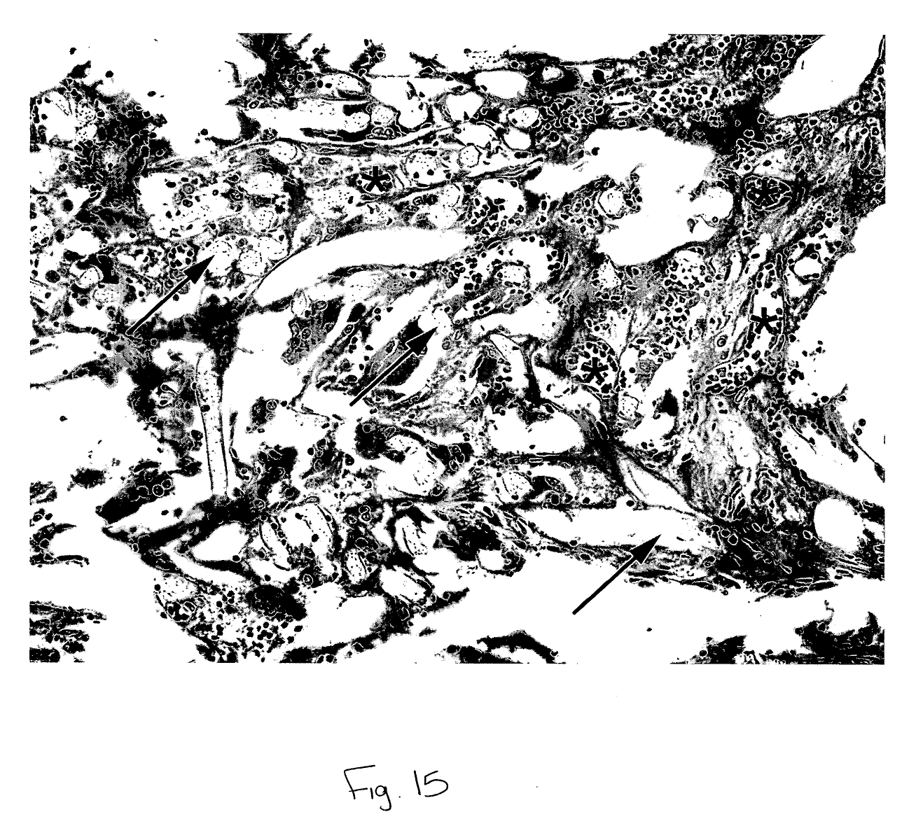

FIG. 7 is a 200× magnification of the FIG. 7 region of FIG. 1 and shows granulation tissue near the edge of new tissue ingrowth with multiple capillaries, as depicted with an asterisk, *, and interlacing Dacron microfilaments, as depicted by arrows. This response was also observed in the human study as depicted in FIG. 15 which is a 200× magnification of a region of human uterine tissue analogous to the region depicted in FIG. 7 of sheep uterine tissue.

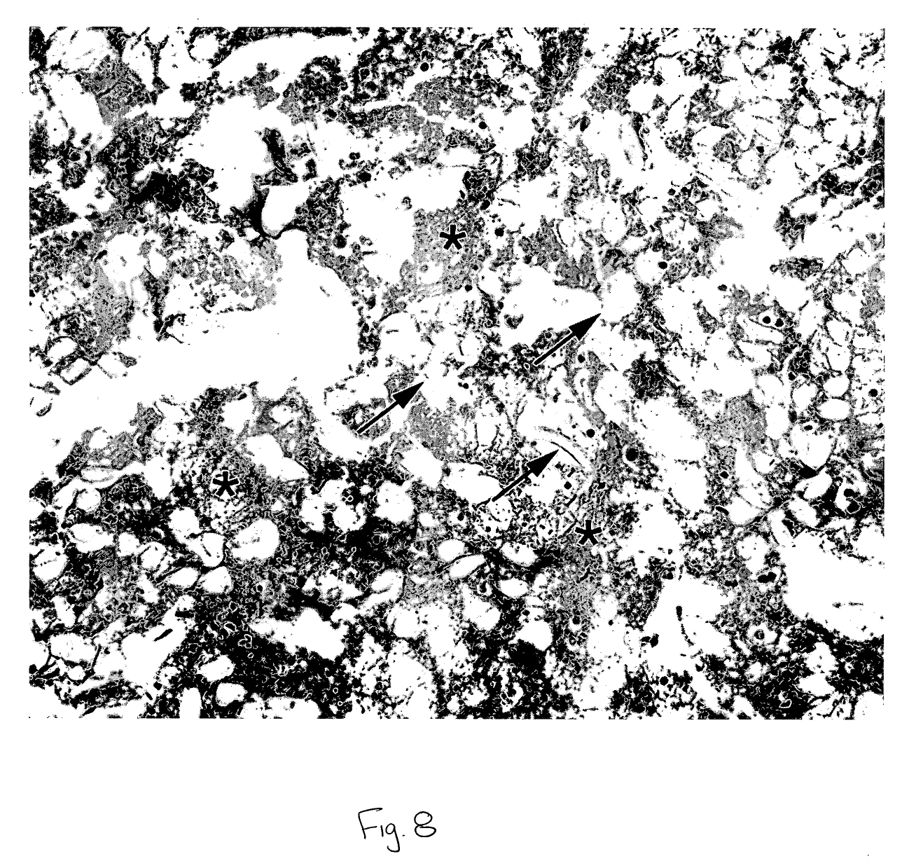

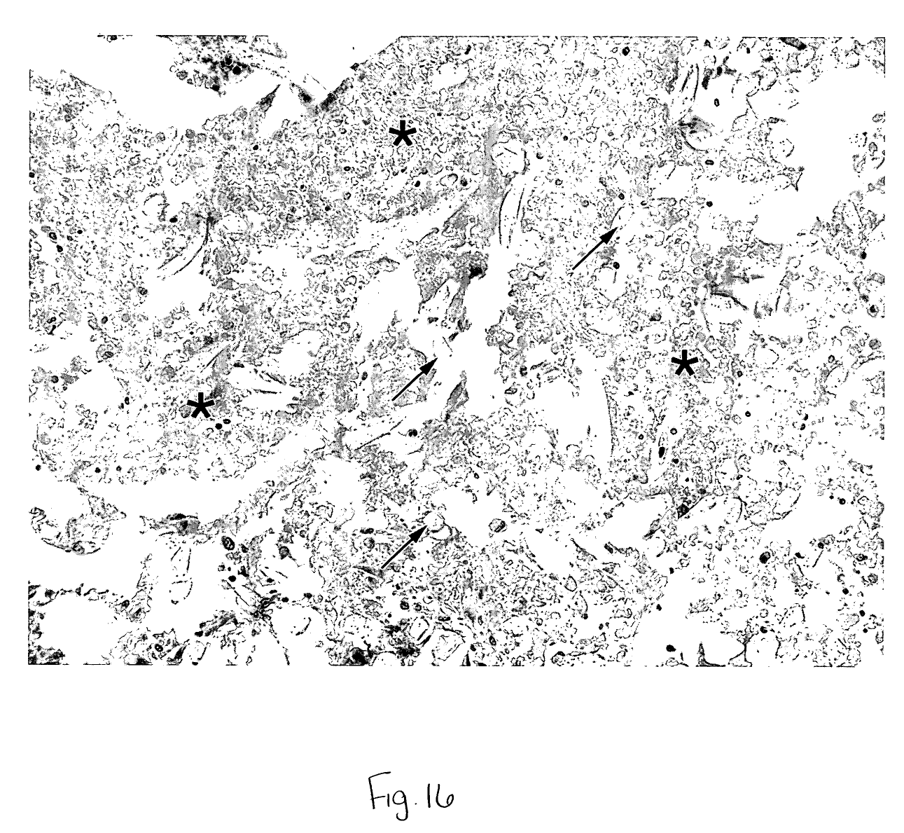

FIG. 8 is a 200× magnification of the FIG. 8 region of FIG. 1 and shows degenerating cells, as evidenced by an asterisk, *, adjacent to the edge of new tissue ingrowth. The arrows identify Dacron microfilaments coursing through this region. This response was also observed in the human study as depicted in FIG. 16 which is a 200× magnification of a region of human uterine tissue analogous to the region depicted in FIG. 8 of sheep uterine tissue.

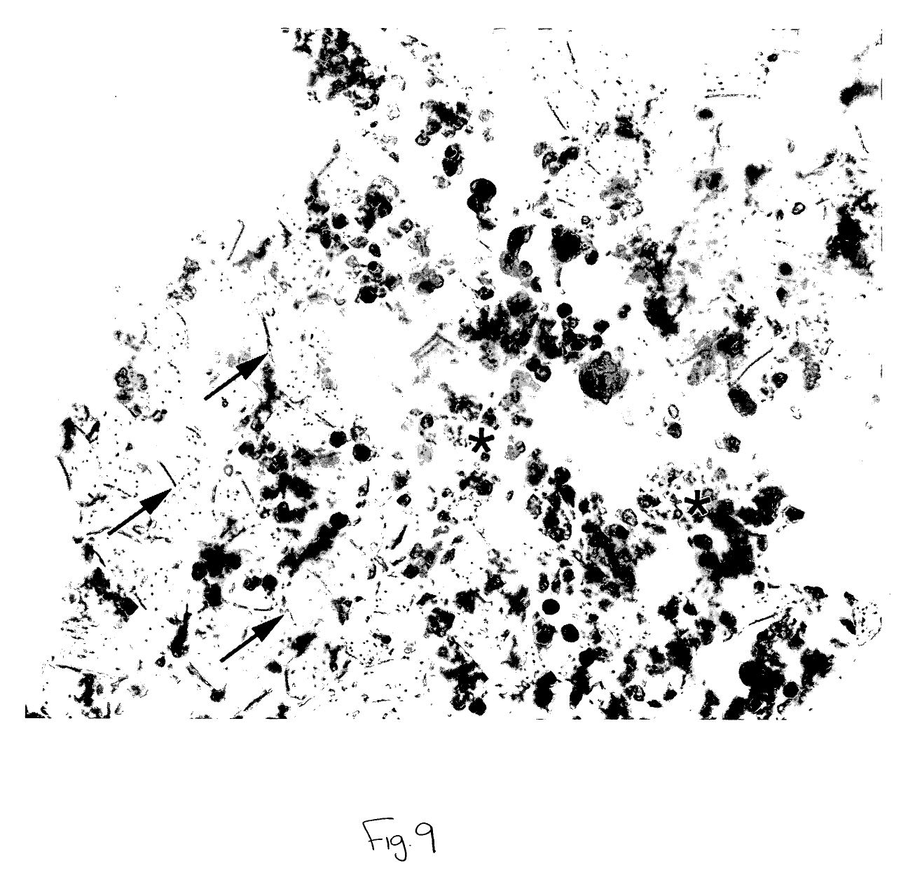

FIG. 9 is a 400× magnification of the FIG. 9 region of FIG. 1 and shows the Dacron fibers in approximation with degenerating cells at the edge of new tissue ingrowth as depicted by the asterisks, *. Dacron microfilaments, as delineated by the arrows, are also present.

As is evident from these figures, the studies show a fibroproliferative reaction that occurs with a bioreactive substance, in this case, Dacron. As such, it confirms the viability of the present invention. That is, it confirms that a bioreactive implant will cause a tissue response that obliterates the normal physiological relationship and function of the endometrium-myometrium which typically leads to uterine bleeding. In this regard, however, it should be noted that the bioreactive aspect of the present invention should not be confused with an inflammatory reaction that might otherwise be encountered with implanted materials. As evidence in the figures presented herein, the tissue response is bioreactive in nature, there being a notable lack of inflammatory cells in any substantial degree.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A method of treating a bleeding pattern in a uterus of a female comprising:
introducing a bioreactive substance into the uterus of a patient;
causing a reaction in said uterus using said bioreactive substance;
wherein said reaction is characterized by substantial elimination of the presence of endometrial cells; invagination of the bioreactive substance by movement of myometrial cells from the myometrium; and substantial adherence of said bioreactive substance to a wall of said uterus; and,
minimizing the formation of inflammatory cells in said uterus.

2. A method according to claim 1, wherein the introducing of a bioreactive substance includes introducing a polyester implant into the uterus.

3. A method according to claim 2, wherein the introducing of a bioreactive substance includes introducing a PET implant into said uterus.

4. A method according to claim 1, wherein the causing of a reaction includes causing a proliferative reaction.

5. A method according to claim 1, wherein the causing of a reaction includes causing a fibroproliferative reaction.

6. A method according to claim 1, wherein the causing of a reaction includes causing an inducement of fibrosis.

7. A method according to claim 1, wherein the causing of a reaction includes causing angiogenesis.

8. A method according to claim 1, wherein the causing of a reaction includes causing fibroblast migration.

9. A method according to claim 1, wherein the causing of a reaction includes causing metaplasia.

10. An implant for treating a uterine bleeding pattern of a female comprising:
a substance sized and shaped for implantation into a uterus of said female;
said substance being biocompatible;
said substance being bioreactive so as to cause a natural infiltration of myometrial tissue into said substance; and,
said substance being substantially non-inflammatory to uterine tissue.

11. An implant as set forth in claim 10, wherein said substance is a mesh.

12. An implant as set forth in claim 10, wherein said substance is polyester.

13. An implant as set forth in claim 12, wherein said polyester is a polyester mesh.

14. An implant as set forth in claim 13, wherein said polyester mesh is a PET.

15. An implant as set forth in claim 10, wherein said substance is an implant comprising a framework covered with a polyester fabric.

16. An implant as set forth in claim 10, wherein said bioreactive substance is a substance that causes a fibroproliferative response.
